(19) United States
(12) Patent Application Publication
(10) Pub. No.: US 2025/0232089 A1
(43) Pub. Date: Jul. 17, 2025

(54) METHOD AND APPARATUS FOR SIMULATING THERMAL STRESS OF CASTING MOLD DURING SERVICE PROCESS, AND STORAGE MEDIUM

(71) Applicant: CITIC Dicastal Co., Ltd., Qinhuangdao City (CN)

(72) Inventors: Xiaoying Ma, Qinhuangdao City (CN); Yuling Lang, Qinhuangdao City (CN); Shuwei Feng, Qinhuangdao City (CN); Kaiqing Wang, Qinhuangdao City (CN); Yacong Zhang, Qinhuangdao City (CN); Haijun Guo, Qinhuangdao City (CN); Dengyao Li, Qinhuangdao City (CN); Qifeng Dai, Qinhuangdao City (CN); Decai Kong, Qinhuangdao City (CN); Ming Yao, Qinhuangdao City (CN); Jian Gao, Qinhuangdao City (CN)

(73) Assignee: CITIC Dicastal Co., Ltd., Qinhuangdao City, HE (CN)

(21) Appl. No.: 19/096,642

(22) Filed: Mar. 31, 2025

(30) Foreign Application Priority Data
Apr. 8, 2024 (CN) 202410416137.9

(51) Int. Cl. G06F 30/23; G06F 30/10; G06F 113/22; G06F 119/08

(52) U.S. Cl. CPC G06F 30/23; G06F 30/10; G06F 2113/22; G06F 2119/08

(57) ABSTRACT

The present invention discloses a method and apparatus for simulating thermal stress of a casting mold during a service process, and a storage medium. The method comprises: importing a pre-built three-dimensional geometric model of the casting mold, processing the geometric model, and then performing grid division to obtain a casting simulation finite element physical model; assigning material parameters, interface parameters, process parameters, and boundary conditions to the finite element physical model to obtain a casting simulation finite element calculation model; performing casting process simulation calculations to obtain casting simulation results such as mold temperature field and stress field, and exporting result data; interpolating a temperature-stress correction factor; multiplying all correction factor data under the same node numbers with mold stress data to obtain final thermal stress distribution data of the mold during the service process.

### TECHNICAL FIELD

The present invention relates to the field of casting numerical simulation, specifically to a method and apparatus for simulating thermal stress of a casting mold during a service process, and a storage medium.

### BACKGROUND

At present, in the casting industry, especially in the field of low pressure and differential pressure casting, product structures face a single-product mass mode, such as wheel and steering knuckle products. These industrial products require casting molds to serve tens of thousands of times during the production process in order to share production costs and enhance the competitiveness of enterprises. However, the complex shapes of parts and the strict process conditions incur complex mold structures. In addition, due to severe degradation of material performance at high temperatures during the service process of molds, the problem of early cracking of the molds is very serious. So far, how to quickly and accurately predict the thermal stress of molds during the casting process has been a major common challenge in the field of casting numerical simulation. Therefore, it is particularly important to accurately predict the thermal stress of casting molds during the service process and use it positively in the product development stage, so as to guide the optimization design of products and processes and reduce the thermal stress of the molds during the service process. As such, early cracking of the molds can be effectively alleviated, the service life of the molds can be prolonged, and the production costs of products can be reduced.

### SUMMARY

To solve the above technical problems, the objective of the present invention is to provide a method and apparatus for simulating thermal stress of a casting mold during a service process, and a storage medium, which can achieve rapid modeling and high-precision and efficient simulation calculation of thermal stress of casting molds during the service process for large and complex automotive parts.

The present invention provides a method for simulating thermal stress of a casting mold during a service process, including:
a step of obtaining a finite element physical model: importing a pre-built three-dimensional geometric model of the casting mold, deleting exhaust channels on fitting surfaces in the three-dimensional geometric model of the casting mold, generating a geometric model of a casting inside the mold through Boolean operation, and performing grid division based on the three-dimensional geometric model of the casting mold and the geometric model of the casting to obtain the three-dimensional geometric model of the casting mold and the geometric model of the casting with grid information as a casting simulation finite element physical model;
a step of obtaining a finite element calculation model: assigning material parameters to components in the casting simulation finite element physical model obtained in the above step, interface heat transfer parameters to contact interfaces in the model, a pressure process parameter to an inlet of a sprue, a cooling heat transfer parameter to a cooling channel surface, and an air boundary heat transfer parameter to an outer surface of the mold, and setting a casting cycle, so as to obtain a casting simulation finite element calculation model;
a step of obtaining casting simulation results: performing solution calculations of 6-10 individual temperature field cycles based on the casting simulation finite element physical model and the casting simulation finite element calculation model obtained in the above steps to obtain first-stage temperature field distribution, exporting results of the first-stage temperature field distribution, inheriting and inputting the results of the first-stage temperature field distribution into 3-5 temperature field and flow field coupled cycles for solution calculations to obtain second-stage temperature field distribution, exporting results of the second-stage temperature field distribution, inheriting and inputting the results of the second-stage temperature field distribution into 1 temperature field, flow field and stress field coupled cycle for solution calculation to obtain third-stage temperature field distribution and stress field distribution, and exporting third-stage temperature field distribution data and stress field distribution data, where the third-stage temperature field distribution data includes all finite element grid node numbers and corresponding temperature values, and the stress field distribution data includes all finite element grid node numbers and corresponding stress values, where all finite element grid nodes of the temperature field and the stress field correspond one to one;
a step of introducing a mold temperature-stress correction factor: introducing a mold temperature-stress correction factor corresponding to the temperature of the mold; and
a step of obtaining final thermal stress distribution data of the mold during the service process: obtaining corresponding mold temperature-stress correction factor distribution data under the third-stage temperature field distribution from the mold temperature-stress correction factor and the third-stage temperature field distribution data, and multiplying all the mold temperature-stress correction factor data under the same node numbers with the third-stage mold stress distribution data to obtain the final thermal stress distribution data of the mold during the service process.

According to other embodiments, the present invention provides an apparatus for simulating thermal stress of a casting mold during a service process, including:
a unit for obtaining a finite element physical model, configured to import a pre-built three-dimensional geometric model of the casting mold, delete exhaust channels on fitting surfaces in the three-dimensional geometric model of the casting mold, generate a geometric model of a casting inside the mold through Boolean operation, and perform grid division based on the three-dimensional geometric model of the casting mold and the geometric model of the casting to obtain the three-dimensional geometric model of the casting mold and the geometric model of the casting with grid information as a casting simulation finite element physical model;
a unit for obtaining a finite element calculation model, configured to assign material parameters to components in the casting simulation finite element physical model obtained by the unit for obtaining a finite element physical model, interface heat transfer parameters to contact interfaces in the model, a pressure process parameter to an inlet of a sprue, a cooling heat transfer parameter to a cooling channel surface, and an air boundary heat transfer parameter to an outer surface of the mold, and set a casting cycle, so as to obtain a casting simulation finite element calculation model;
a unit for obtaining casting simulation results, configured to perform solution calculations of 6-10 individual temperature field cycles based on the casting simulation finite element physical model and the casting simulation finite element calculation model obtained by the above units to obtain first-stage temperature field distribution, export results of the first-stage temperature field distribution, inherit and input the results of the first-stage temperature field distribution into 3-5 temperature field and flow field coupled cycles for solution calculations to obtain second-stage temperature field distribution, export results of the second-stage temperature field distribution, inherit and input the results of the second-stage temperature field distribution into 1 temperature field, flow field and stress field coupled cycle for solution calculation to obtain third-stage temperature field distribution and stress field distribution, and export third-stage temperature field distribution data and stress field distribution data, where the third-stage temperature field distribution data includes all finite element grid node numbers and corresponding temperature values, and the stress field distribution data includes all finite element grid node numbers and corresponding stress values, wherein all finite element grid nodes of the temperature field and the stress field correspond one to one;
a unit for introducing a mold temperature-stress correction factor, configured to introduce a mold temperature-stress correction factor corresponding to the temperature of the mold; and
a unit for obtaining final thermal stress distribution data of the mold during the service process, configured to obtain corresponding mold temperature-stress correction factor distribution data under the third-stage temperature field distribution from the mold temperature-stress correction factor and the third-stage temperature field distribution data, and multiply all the mold temperature-stress correction factor data under the same node numbers with the third-stage mold stress distribution data to obtain final thermal stress distribution data of the mold during the service process.

According to other embodiments, the present invention provides an electronic device, including a memory and a processor, where the memory stores a computer program capable of running on the processor, and the processor executes the program to implement the method for simulating thermal stress of a casting mold during a service process.

According to other embodiments, the present invention provides a storage medium that is a computer-readable storage medium storing a computer program, where the program, when executed by a processor, implements the method for simulating thermal stress of a casting mold during a service process.

Beneficial effects of the present invention are as follows:
1. Due to severe mechanical performance degradation of mold materials at high temperatures, the risk of mold cracking and failure cannot be evaluated solely based on the magnitude of thermal stress during the service process under high temperature conditions, but influencing factors of mold temperature need to be considered. The present invention innovatively introduces the mold temperature-stress correction factor to correct the stress field of the mold, whereby the magnitude distribution of thermal stress of the mold is re-calculated to evaluate the risk of mold cracking. A brand-new method and process for simulating thermal stress of a mold, considering temperature factors, are developed to solve the problem of difficult modeling and calculation of thermal stress in large and complex automotive part molds. As engineers and research and development personnel use this method for optimization design in product design and process design stages, the thermal stress of the mold during the service process is reduced to extend the service life of the mold.
2. The present invention greatly shortens the development cycle of new products. By using this method in the product design and process design stages, the trial and error cycle and cost of later production trial are advanced to the development stage. By replacing production trial with simulation, the number of mold repairs in later processes is reduced, and the development cycle and cost of new products are greatly reduced.
3. The present invention greatly solves the cracking problem of mass-produced molds. By using this method on mass-produced cracked molds, it can accurately identify mold stress concentration caused by unreasonable design of rounded corners, unreasonable design of mold structures, unreasonable design of cooling processes, etc. Through shape and process optimization for identified problems, mold structures can be improved to adapt to harsh service conditions, thereby prolonging the service life of molds.

### BRIEF DESCRIPTION OF THE DRAWINGS

Hereinafter, embodiments of the present invention will be described in detail with reference to the accompanying drawings.

FIG. 1 is a flowchart of a method for simulating thermal stress of a casting mold during a service process in an embodiment of the present invention.

FIG. 2 is a schematic diagram of a casting simulation finite element physical model in an embodiment of the present invention.

FIG. 3 is a schematic diagram of a temperature field in a third stage in casting simulation results in an embodiment of the present invention.

FIG. 4 is a schematic diagram of a stress field of a mold in casting simulation results in an embodiment of the present invention.

FIG. 5 is a schematic diagram before product shape optimization in an embodiment of the present invention.

FIG. 6 is a schematic contrast diagram after product shape optimization in an embodiment of the present invention.

FIG. 7 is a final schematic diagram of a mold stress field before product shape optimization in an embodiment of the present invention.

FIG. 8 is a schematic final contrast diagram of a mold stress field after product shape optimization in an embodiment of the present invention.

### EXPLANATION OF REFERENCE NUMERALS

1—upper mold; 2—sprue spreader; 3—exhaust insert; 4—slider; 5—casting; 6—sprue cup; 7—sprue; 8—sprue bush; 9—lower mold.

### DETAILED DESCRIPTION

Hereinafter, exemplary embodiments of the present invention will be described in detail with reference to the accompanying drawings. The exemplary embodiments described below and illustrated in the accompanying drawings are intended to teach the principle of the present invention, enabling those skilled in the art to implement and use the present invention in various environments and for various applications. Therefore, the scope of protection of the present invention is defined by the appended claims, and the exemplary embodiments are not intended and should not be considered as limiting descriptions of the scope of protection of the present invention. Moreover, for ease of description, the dimensions of various portions shown in the accompanying drawings are not necessarily drawn according to actual proportional relationships. For orientation descriptions, the orientation or position relationship indicated by terms, for example, up, down, left, right, top, and bottom, are based on the orientation or position relationships shown in the accompanying drawings, only for the convenience of describing the present invention and simplifying the description, and do not indicate or imply that the apparatus or element referred to must have a specific orientation and be constructed and operated in a specific orientation. Therefore, the terms cannot be understood as a limitation of the present invention. Unless otherwise specified, the sequence and numerical values of the components and assembly steps described in the embodiments do not limit the scope of the present invention. Moreover, any numerical range stated herein is intended to include all sub ranges contained therein, and the numerical range represented by “numerical A to numerical B” refers to a range that includes endpoint values A and B. Those skilled in the art can understand that the terms “first”, “second”, “step”, etc. in the present invention are only used to distinguish different steps, devices, or modules, and do not represent any specific technical meaning, nor do they indicate a necessary logical order between them. For example, steps two and three can be exchanged or parallelized.

### Embodiment 1

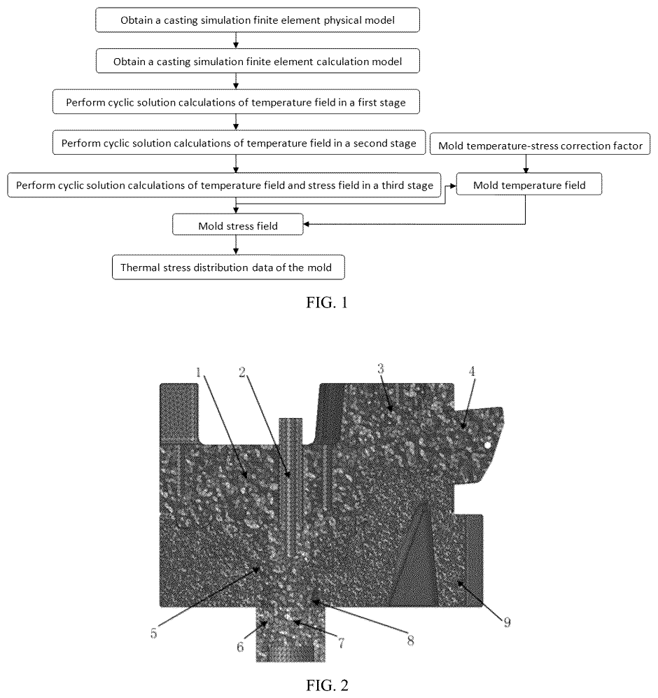

With reference to FIG. 1 to FIG. 8, a first embodiment of the present invention provides a method and apparatus for simulating thermal stress of a casting mold during a service process, and a computer-readable storage medium. Specifically, the method and apparatus for simulating thermal stress of a casting mold during a service process can be implemented by a device for simulating thermal stress of a casting mold during a service process (hereinafter referred to as simulation device), particularly implemented by one or more processors in the simulation device executing programs. In addition, the computer-readable storage medium is a non-transient storage medium that stores a computer program used for implementing the method. Hereinafter, with reference to FIG. 1, a simulation device is used as an example for explaining the method for simulating thermal stress of a casting mold during a service process. The method includes at least the following steps.

### Step 1: Obtain a Casting Simulation Finite Element Physical Model.

In this embodiment, the simulation device first imports a pre-built three-dimensional geometric model of the casting mold. To ensure that various portions of the mold fit tightly without gaps or interference, exhaust channels on fitting surfaces in the three-dimensional geometric model of the casting mold are deleted. A geometric model of a casting inside the mold is generated through Boolean operation. Then, grid division is performed based on the three-dimensional geometric model of the casting mold and the geometric model of the casting, where the three-dimensional geometric model of the casting mold and the geometric model of the casting with grid information serve as the casting simulation finite element physical model (as shown in FIG. 2, for example). It should be noted that, as shown in FIG. 2, the casting simulation finite element physical model generally includes an upper mold 1, a sprue spreader 2, an exhaust insert 3, a slider 4, a lower mold 9, a sprue bush 8, a sprue cup 6, a sprue 7, a casting 5, etc. In case of many cracks in the lower mold during the actual service process of the mold, and in order to facilitate device calculation and solution, the grid size of the lower mold of great concern is set to 2-3 mm, and the grid sizes of the casting 5 and other mold portions are set to 6-10 mm, thereby ensuring the calculation accuracy of the lower mold 9 and satisfying the overall calculation efficiency. The divided grids are tetrahedral single-node grids, and the number of grids in this embodiment is approximately 3 million.

### Step 2: Obtain a Casting Simulation Finite Element Calculation Model.

Based on the above casting simulation finite element physical model, firstly, material parameters of components of the model are imported into the device for material assignment. For example, an H13 steel material is assigned to the upper mold 1, the lower mold 9, the sprue spreader 2, the exhaust insert 3, the slider 4, and the sprue bush 8, respectively; a ceramic material is assigned to the sprue cup 6; and an A356 aluminum alloy material is assigned to the casting 5 and the sprue 7. Secondly, heat transfer parameters of interfaces are imported into the device, where the interfaces include contact interfaces between the casting 5 and the upper and lower molds (the upper mold 1 and the lower mold 9), a contact interface between the upper mold 1 and the lower mold 9, a contact interface between the sprue spreader 2 and the upper mold 1, a contact interface between the exhaust insert 3 and the upper mold 1, contact interfaces between the slider 4 and the upper and lower molds, a contact interface between the sprue bush 8 and the lower mold 9, and other contact interfaces in the model; and interface parameters are assigned to the interfaces where various components of the model contact each other. Next, a pressure process parameter is assigned to an inlet of the sprue 7. Finally, a cooling heat transfer parameter is assigned to a cooling channel surface of the mold, an air boundary heat transfer parameter is assigned to an outer surface of the mold, and a casting cycle is set, so as to obtain the casting simulation finite element calculation model.

### Step 3: Obtain Casting Simulation Results.

It should be noted that, due to severe cracking of the lower mold, this embodiment describes only the lower mold 9, and other mold result analysis methods are consistent with this.

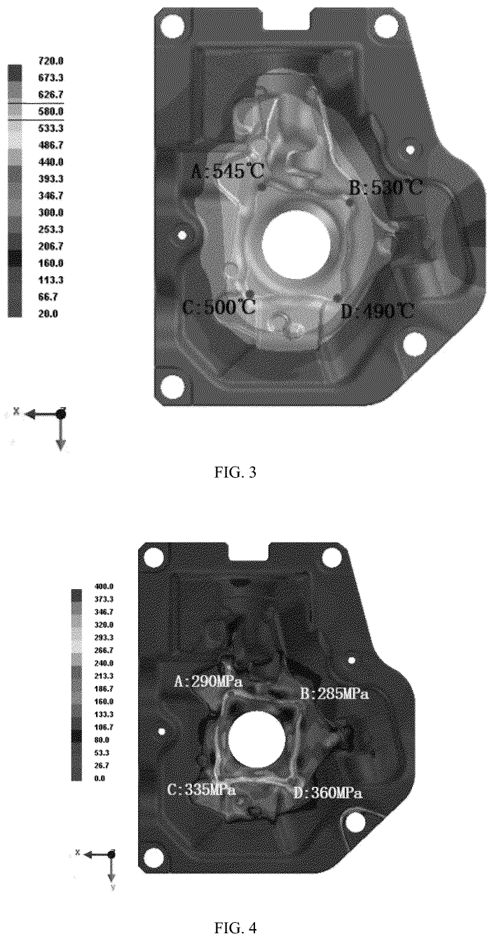

Based on the finite element models obtained through steps 1 and 2 above, simulation in a first stage (6-10 individual temperature field cycles) is first solved to calculate a temperature field distribution of the lower mold at the end of the cycles. In this case, the mold is in a pre-heating stage and has not reached a temperature of stable production heat balance. The temperature field distribution results of the lower mold calculated in the first stage are exported, inherited and inputted into a second stage (3-5 temperature field and flow field coupled cycles) for solution calculations to obtain a temperature field distribution of the lower mold at the end of the cycles. After 9-15 cycles, the temperature of the mold is almost stable. The temperature field of the mold can represent the actual temperature of the mold after heat balance during final stable production. The temperature field distribution results of the lower mold calculated in the second stage are exported, inherited and inputted into a third stage (1 temperature field, flow field and stress field coupled cycle) for solution calculations to obtain a temperature field distribution (as shown in FIG. 3) and a stress field distribution (as shown in FIG. 4) of the lower mold at the end of the cycle. Then, the mold is in a heat balance state, the temperature of the mold reaches an actual temperature after heat balance during stable production, and the thermal stress caused by temperature gradient changes can represent a stress state under stable production during the final service process of the mold. From the stress field of the mold in FIG. 4, it can be preliminarily determined that peak positions of thermal stress in the mold are 335 MPa at point C and 360 MPa at point D, and risk positions of mold cracking and failure are at points C and D. The stress values at points A and B are relatively low, 290 MPa and 285 MPa, but from the temperature field distribution in FIG. 3, the temperatures at points A and B are higher, and the stress values at the two points will be amplified by weighting a temperature-stress factor. Therefore, this embodiment focuses on the stress simulation results of positions of points A, B, C, and D (where points A, B, C, and D represent finite element grid node numbers at 4 positions on the surface of a mold cavity, respectively);

Temperature field distribution data and stress field distribution data of the mold in this state are exported, where the temperature field distribution data is all finite element grid node numbers and corresponding temperature values, and the stress field distribution data is all finite element grid node numbers and corresponding stress values, where all finite element grid nodes of the temperature field and the stress field are consistent, that is, all the finite element grid node numbers correspond one to one. All the temperature field distribution data and the stress field distribution data are stored in an excel of the device for later direct calling with Python compiled scripts. In this embodiment, the temperature values at points A, B, C, and D are 545° C., 530° C., 500° C., and 490° C., and the corresponding initial stress values at the four points are 290 MPa, 285 MPa, 335 MPa, and 360 MPa.

Step 4: Introduce a mold temperature-stress correction factor for correcting the thermal stress of the mold. Specifically, based on the phenomenon of material performance degradation of mold steel at high temperatures, the mold temperature-stress correction factor is positively correlated with the temperature of the mold. Therefore, the mold temperature-stress correction factor is introduced corresponding to the temperature of the mold. More specifically, for example, the correction factor when the temperature of the mold is 100° C. is 0.6, the correction factor when the temperature of the mold is 200° C. is 0.7, the correction factor when the temperature of the mold is 300° C. is 0.8, the correction factor when the temperature of the mold is 400° C. is 0.9, the correction factor when the temperature of the mold is 500° C. is 1.0, the correction factor when the temperature of the mold is 550° C. is 1.1, the correction factor when the temperature of the mold is 600° C. is 1.2, and the correction factors at other temperature intervals are calculated by linear interpolation. In this embodiment, the temperature-stress correction factors for points A, B, C, and D are 1.09, 1.06, 1, and 0.99, respectively.

Step 5: Obtain corresponding mold temperature-stress correction factor distribution data under the temperature field distribution from the temperature-stress correction factor obtained in step 4 and the temperature field distribution data obtained in the third stage of step 3, and multiply all the mold temperature-stress correction factor data under the same node numbers with the mold stress data obtained in step 3 to obtain final thermal stress distribution data of the mold during the service process. Further, positions with thermal stress values close to or greater than 300 MPa can be selected from the final thermal stress distribution data of the mold during the service process as mold cracking risk positions. According to the production statistics of the service mold, the risk of mold cracking is relatively high when the thermal stress of the service mold is close to or greater than 300 MPa. In this embodiment, the final thermal stresses of the mold during the service process at points A, B, C, and D are 316 MPa, 302 MPa, 335 MPa, and 356 MPa, respectively, and the four positions are identified as mold cracking risk positions.

Prior to the use of the present invention, conventional simulation methods can identify only two positions, point C and point D, as mold cracking risk positions, while in this embodiment of the present invention, more mold cracking risk positions are identified, and structural optimization can be further performed on the identified mold cracking risk positions to alleviate mold failure. In the actual production process, the mold indeed cracks in the 4 areas.

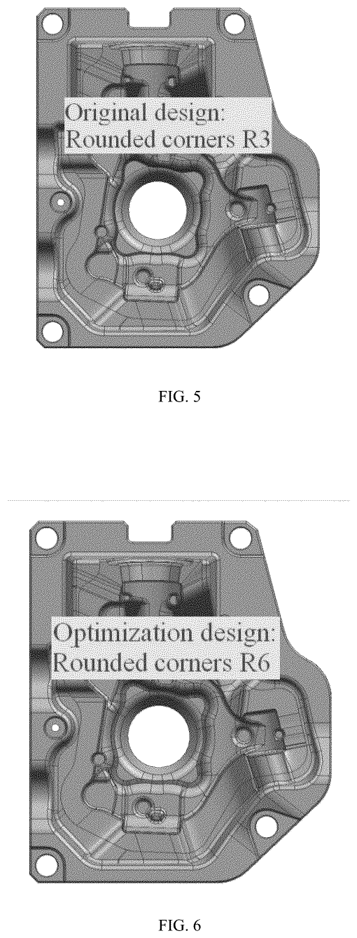

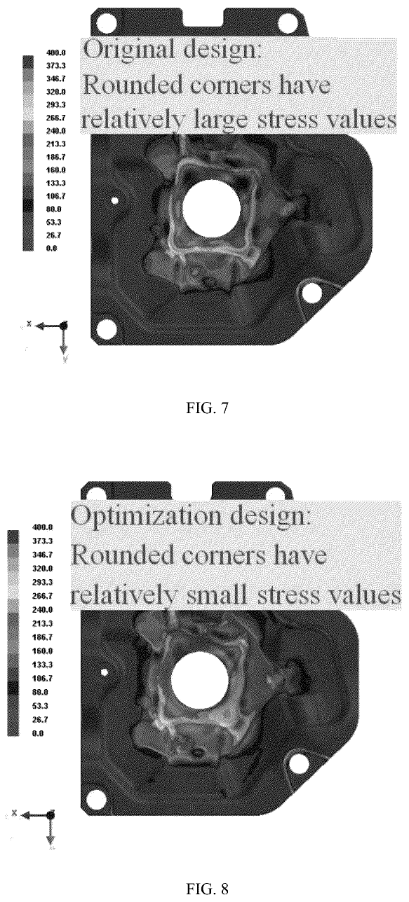

In this embodiment, points A, B, C, and D are identified as mold cracking risk positions. After analysis, it is found that the rounded corners at the four positions are relatively small, i.e. R3mm (as shown in FIG. 5). Later, after structural optimization of the mold, the rounded corners are optimized to R6mm (as shown in FIG. 6). After optimization, the simulation analysis on the thermal stress of the mold shows that the thermal stress of the mold during the service process decreases to varying degrees (as shown in FIG. 7 and FIG. 8), which greatly helps prolong the service life of the mold in the later stage.

The above explains a method for simulating thermal stress of a casting mold during a service process through Embodiment 1. Those skilled in the art can clearly understand that an apparatus for simulating thermal stress of a casting mold during a service process has various units for implementing the above steps and can achieve the same technical effects. Moreover, by reading and executing a program stored in a storage medium for implementing the method, a processor can also achieve the same technical effects.

1. A method for simulating thermal stress of a casting mold during a service process, comprising:
a step of obtaining a finite element physical model: importing a pre-built three-dimensional geometric model of the casting mold, deleting exhaust channels on fitting surfaces in the three-dimensional geometric model of the casting mold, generating a geometric model of a casting inside the mold through Boolean operation, and performing grid division based on the three-dimensional geometric model of the casting mold and the geometric model of the casting to obtain the three-dimensional geometric model of the casting mold and the geometric model of the casting with grid information as a casting simulation finite element physical model;
a step of obtaining a finite element calculation model: assigning material parameters to components in the casting simulation finite element physical model obtained in the above step, interface heat transfer parameters to contact interfaces in the model, a pressure process parameter to an inlet of a sprue, a cooling heat transfer parameter to a cooling channel surface, and an air boundary heat transfer parameter to an outer surface of the mold, and setting a casting cycle, so as to obtain a casting simulation finite element calculation model;
a step of obtaining casting simulation results: performing solution calculations of 6-10 individual temperature field cycles based on the casting simulation finite element calculation model obtained in the above step to obtain first-stage temperature field distribution, exporting results of the first-stage temperature field distribution, inheriting and inputting the results of the first-stage temperature field distribution into 3-5 temperature field and flow field coupled cycles for solution calculations to obtain second-stage temperature field distribution, exporting results of the second-stage temperature field distribution, inheriting and inputting the results of the second-stage temperature field distribution into 1 temperature field, flow field and stress field coupled cycle for solution calculation to obtain third-stage temperature field distribution and stress field distribution, and exporting third-stage temperature field distribution data and stress field distribution data, wherein the third-stage temperature field distribution data comprises all finite element grid node numbers and corresponding temperature values, and the stress field distribution data comprises all finite element grid node numbers and corresponding stress values, wherein all finite element grid nodes of the temperature field and the stress field correspond one to one;
a step of introducing a mold temperature-stress correction factor: introducing a mold temperature-stress correction factor corresponding to the temperature of the mold; and
a step of obtaining final thermal stress distribution data of the mold during the service process: obtaining corresponding mold temperature-stress correction factor distribution data under the third-stage temperature field distribution from the mold temperature-stress correction factor and the third-stage temperature field distribution data, and multiplying all the mold temperature-stress correction factor data under the same node numbers with the third-stage mold stress distribution data to obtain the final thermal stress distribution data of the mold during the service process,
wherein
in the step of introducing a mold temperature-stress correction factor, the mold temperature-stress correction factor when the temperature of the mold is 100° C. is 0.6, the mold temperature-stress correction factor when the temperature of the mold is 200° C. is 0.7, the mold temperature-stress correction factor when the temperature of the mold is 300° C. is 0.8, the mold temperature-stress correction factor when the temperature of the mold is 400° C. is 0.9, the mold temperature-stress correction factor when the temperature of the mold is 500° C. is 1.0, the mold temperature-stress correction factor when the temperature of the mold is 550° C. is 1.1, the mold temperature-stress correction factor when the temperature of the mold is 600° C. is 1.2, and the mold temperature-stress correction factors at other temperature intervals are calculated by linear interpolation.

2. The method for simulating the thermal stress of the casting mold during the service process according to claim 1, wherein
in the step of obtaining final thermal stress distribution data of the mold during the service process, positions with thermal stress values greater than 300 MPa are further selected from the final thermal stress distribution data as mold cracking risk positions.

3. The method for simulating the thermal stress of the casting mold during the service process according to claim 1, wherein
in the step of obtaining a finite element physical model, the grid size of a lower mold is set to 2-3 mm, and the grid sizes of the casting and other mold portions are set to 6-10 mm.

4. An apparatus for simulating thermal stress of a casting mold during a service process, comprising:
a unit for obtaining a finite element physical model, configured to import a pre-built three-dimensional geometric model of the casting mold, delete exhaust channels on fitting surfaces in the three-dimensional geometric model of the casting mold, generate a geometric model of a casting inside the mold through Boolean operation, and perform grid division based on the three-dimensional geometric model of the casting mold and the geometric model of the casting to obtain the three-dimensional geometric model of the casting mold and the geometric model of the casting with grid information as a casting simulation finite element physical model;
a unit for obtaining a finite element calculation model, configured to assign material parameters to components in the casting simulation finite element physical model obtained by the above unit, interface heat transfer parameters to contact interfaces in the model, a pressure process parameter to an inlet of a sprue, a cooling heat transfer parameter to a cooling channel surface, and an air boundary heat transfer parameter to an outer surface of the mold, and set a casting cycle, so as to obtain a casting simulation finite element calculation model;
a unit for obtaining casting simulation results, configured to perform solution calculations of 6-10 individual temperature field cycles based on the casting simulation finite element calculation model obtained by the above unit to obtain first-stage temperature field distribution, export results of the first-stage temperature field distribution, inherit and input the results of the first-stage temperature field distribution into 3-5 temperature field and flow field coupled cycles for solution calculations to obtain second-stage temperature field distribution, export results of the second-stage temperature field distribution, inherit and input the results of the second-stage temperature field distribution into 1 temperature field, flow field and stress field coupled cycle for solution calculation to obtain third-stage temperature field distribution and stress field distribution, and export third-stage temperature field distribution data and stress field distribution data, wherein the third-stage temperature field distribution data comprises all finite element grid node numbers and corresponding temperature values, and the stress field distribution data comprises all finite element grid node numbers and corresponding stress values, wherein all finite element grid nodes of the temperature field and the stress field correspond one to one;
a unit for introducing a mold temperature-stress correction factor, configured to introduce a mold temperature-stress correction factor corresponding to the temperature of the mold; and
a unit for obtaining final thermal stress distribution data of the mold during the service process, configured to obtain corresponding mold temperature-stress correction factor distribution data under the third-stage temperature field distribution from the mold temperature-stress correction factor and the third-stage temperature field distribution data, and multiply all the mold temperature-stress correction factor data under the same node numbers with the third-stage mold stress distribution data to obtain final thermal stress distribution data of the mold during the service process,
wherein
in the unit for introducing a mold temperature-stress correction factor, the mold temperature-stress correction factor when the temperature of the mold is 100° C. is 0.6, the mold temperature-stress correction factor when the temperature of the mold is 200° C. is 0.7, the mold temperature-stress correction factor when the temperature of the mold is 300° C. is 0.8, the mold temperature-stress correction factor when the temperature of the mold is 400° C. is 0.9, the mold temperature-stress correction factor when the temperature of the mold is 500° C. is 1.0, the mold temperature-stress correction factor when the temperature of the mold is 550° C. is 1.1, the mold temperature-stress correction factor when the temperature of the mold is 600° C. is 1.2, and the mold temperature-stress correction factors at other temperature intervals are calculated by linear interpolation.

5. The apparatus for simulating the thermal stress of the casting mold during the service process according to claim 4, wherein
in the unit for obtaining final thermal stress distribution data of the mold during the service process, positions with thermal stress values greater than 300 MPa are further selected from the final thermal stress distribution data as mold cracking risk positions.

6. The apparatus for simulating the thermal stress of the casting mold during the service process according to claim 4, wherein
in the unit for obtaining a finite element physical model, the grid size of a lower mold is set to 2-3 mm, and the grid sizes of the casting and other mold portions are set to 6-10 mm.

7. A storage medium that is a computer-readable storage medium storing a computer program, wherein the program, when executed by a processor, implements the method according to claim 1.
